(19) United States
(12) Patent Application Publication
(10) Pub. No.: US 2024/0175008 A1
(43) Pub. Date: May 30, 2024

(54) SYNTHESIZING CHEMICAL LIBRARIES USING DIGITAL MICROFLUIDICS

(71) Applicant: FemtoFluidics, Inc., Excelsior, MN (US)

(72) Inventors: Dung HOANG, Burnsville, MN (US); Zachary XIONG, Minneapolis, MN (US)

(21) Appl. No.: 18/525,013

(22) Filed: Nov. 30, 2023

Related U.S. Application Data
(60) Provisional application No. 63/385,436, filed on Nov. 30, 2022.

(51) Int. Cl. C12N 15/10; B01L 3/00

(52) U.S. Cl. CPC C12N 15/1065; B01L 3/502792; B01L 2300/0645; B01L 2300/0663; B01L 2300/161; B01L 2400/0427

(57) ABSTRACT

Various systems and methods that synthesize chemical libraries and/or screen chemical compounds are described. The technology utilizes a digital microfluidic (DMF) device to perform various operations, such as the synthesizing and/or screening of chemical libraries. The DMF device can be configured to operate on small droplets (e.g., ˜nanoliter sizes), to operate on many droplets at once (e.g., using a large grid of electrodes), and/or to operate quickly when moving, merging, splitting, mixing, or otherwise manipulating droplets during the synthesis/screening of inputs/compounds.

### CROSS-REFERENCE TO RELATED APPLICATIONS

This application claims priority to U.S. Provisional Patent Application No. 63/385,436, filed on Nov. 30, 2022, entitled ELECTRONIC DEVICE FOR SYNTHESIZING DNA-ENCODED LIBRARIES, which is hereby incorporated by reference in its entirety.

### BACKGROUND

Using large drug or chemical libraries, pharmaceutical companies can screen compounds to find drugs much more efficiently than ever before. The industry has invested in complex infrastructure for the task of creating these libraries, in the form of liquid-handling robotics.

The creation of these libraries involves combinatorial chemistry, which can create a library of different chemical compounds by systematically attaching different building blocks in different combinations to form the compounds. For example, an entity may utilize such techniques by synthesizing DNA “barcodes” and attaching them to compounds to form DNA-encoded libraries (DELs).

Conventional generation of DELs is expensive and time consuming, due to the time and resources required. For example, such processes often utilize liquid-handling robots to bring together droplets containing different building blocks in millions of different combinations, attaching a unique DNA barcode to each combination. Such DELs (or other libraries) may contain millions, billions, or even trillions of entries (e.g., unique compounds).

It follows that a larger library can lead to a greater chance of finding a specific molecule that works as an intended drug. However, such conventional resources (e.g., mechanical robots, pressure-based fluids, acoustic-based techniques) are expensive to build and maintain and cannot perform combinatorial chemistry at sufficient speed or at sufficient scale to build such large libraries. These and other problems exist with respect to conventional synthesis of chemical libraries, such as DELs.

### BRIEF DESCRIPTION OF THE DRAWINGS

Embodiments of the present technology will be described and explained through the use of the accompanying drawings.

FIG. 1 is a block diagram illustrating a suitable environment for synthesizing chemical libraries using digital microfluidics.

FIG. 2 is a block diagram illustrating an example digital microfluidic (DMF) device for synthesizing chemical compounds from chemical inputs.

FIGS. 3A-3D are block diagrams that illustrate different layers of an example DMF device.

FIG. 4 is a diagram illustrating an array of electrodes for use with a DMF device.

FIG. 5 is a diagram illustrating another array of electrodes for use with a DMF device.

FIG. 6 is a flow diagram illustrating a method for creating a chemical library using a DMF device.

FIGS. 7A-7B are diagrams illustrating an electrowetting orchestration of chemical inputs using a DMF device.

FIG. 8 is a flow diagram illustrating a method for screening chemical compounds using a DMF device.

FIG. 9 is a diagram illustrating an electrowetting orchestration of chemical compounds using a DMF device.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### Overview

Various systems and methods that synthesize chemical libraries and/or screen chemical compounds using electronic-based techniques are described. The systems and methods, in some embodiments, employ a digital microfluidic (DMF) device to perform liquid handling (e.g., moving, combining, splitting, and so on) of drops, such as drops that contain chemical inputs and/or chemical compounds.

As described herein, conventional techniques of synthesizing libraries have reached effective limits imposed by speed (and possibly cost). The technology described herein, however, is not constrained by such limits, as it employs digital microfluidics, which can synthesize large amounts of chemical compounds using relatively smaller volumes of inputs (e.g., chemicals or reagents).

For example, the technology synthesizes compounds on an electronic grid (e.g., via a printed circuit board, or PCB), which does not include moving or mechanical parts or components. The systems and methods manipulate liquids/drops via electrowetting, which utilizes electric charges to move drops across a grid or array of electrodes.

Thus, the technology provides a DMF device adapted or configured to receive chemical inputs, synthesize the inputs into chemical compounds via electrowetting orchestration of the inputs across or over an array of electrodes (e.g., via voltage switching between neighboring electrodes), and output the compounds to a chemical library.

Further, in some embodiments, the DMF device is controlled by a control system, which choreographs the moving drops (with respect to one another) during the synthesis of the chemical library. Thus, the technology can implement a large-scale array of electrodes e.g., 100×100, 1000×1000, or larger) when building a library.

Therefore, in various embodiments, the technology described herein can move smaller drops (or droplets), can move more drops, and/or can move drops faster than conventional techniques. In doing so, the technology can realize the synthesis of large chemical libraries (e.g., DELs) that include millions or billions of entries, among other benefits. Further, the technology can also be utilized to screen the entries of the chemical libraries, providing additional enhancements to entities employing the technology for drug discovery and other research activities.

Various embodiments of the technology will now be described. The following description provides specific details for a thorough understanding and an enabling description of these embodiments. One skilled in the art will understand, however, that these embodiments may be practiced without many of these details. Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of Synthesizing a Chemical Library Using a Digital Microfluidic (DMF) Device

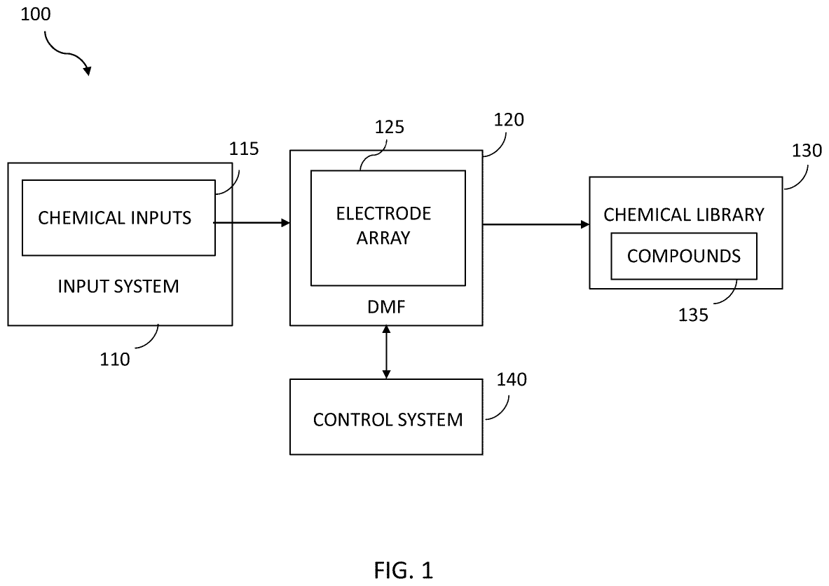

The technology described herein is directed, in some embodiments, to a digital microfluidic (DMF) device that synthesizes a chemical library, such as a DNA-encoded library (DEL). FIG. 1 is a block diagram illustrating a suitable environment 100 for synthesizing chemical libraries using digital microfluidics.

A DMF device 120 is coupled to an input system 110, which provides chemical inputs 115 (e.g., reagents in liquid form, such as drops or droplets). In some cases, the input system 110 includes an automated system that loads the chemical inputs 115 to the DMF device 120 via microfluidic channels or microfluidic tubes. For example, the input system 110 may store or contain the chemical inputs 115 in a microplate (or microtiter plate, microwell plate, or multiwell).

In some cases, the channels/tubes may include (pressure-based) microvalves that control the flow of liquid and pumps that deliver specific volumes of the chemical inputs 115 (e.g., out of a microplate). In some cases, the channels/tubes may include a microfluidics multiplexer that sorts or selects the chemical inputs (from multiple available inputs) to provide or dispense to the DMF device 120.

The DMF device 120 includes an electrode array 125, which can be provided or supported by a printed circuit board (PCB) or other similar substrate. As described herein, the electrode array 125 may include multiple electrodes of a certain geometry, such as electrodes having a triangular shape, electrodes having a hexagonal shape, electrodes having more intricate edge patterns, and so on.

The DMF device 120 synthesizes chemical compounds 135 from the chemical inputs 115, and outputs the chemical compounds 135 to a chemical library 130 coupled to or associated with the DMF device 120. For example, the chemical library 130 and/or the DMF device 120 may include an automated system (e.g., having microfluidic channels/tubes, as described herein) that offloads the chemical compounds to the chemical library 130, such as to one or more microplates.

To synthesize the chemical compounds 135, the DMF device 120, via the electrode array 125, employs electrowetting orchestration to move, split, or otherwise manipulate the chemical inputs 115 within the DMF device 120. For example, an electric field is set at each electrode of the electrode array 125 via an applied voltage, which can either be turned on or off. A drop/droplet rests on a hydrophobic surface above the electrode array 125. The drop moves by turning off the electric field at an electrode where the drop is located while simultaneously turning on the electric field at an adjacent or neighboring electrode (causing the surface above the adjacent electrode to become hydrophilic due to the applied field). The drop flattens when the electric field is applied and is pulled over to the adjacent electrode (e.g., moves to the next electrode).

Thus, the DMF device 120 utilizes digital microfluidics to employ electrowetting and control the wettability of a hydrophobic surface on which the drops sit and move. The drops bead due to surface tension without an applied field, and the application of the electric field flattens or wets them, causing them to move across the electrode array 125.

A control system 140 can implement various movement patterns or orchestrations of drops/droplets across the electrode array 125 when synthesizing the chemical library 130 and/or performing other actions or operations (e.g., moving chemical compounds 130 to specific electrodes for screening or other analyses). Thus, the control system 140, part of a computing system or a machine learning (ML) system, may control the electric fields at each of the electrodes of the electrode array 125 to orchestrate the movement of chemical inputs 114 (or chemical compounds 135) along various paths of the electrode array 125, to specific sections or zones of the electrode array 125, and so on. The DMF device 120, being controlled by the control system 140, can perform various operations with respect to drops, such as dispensing of drops, moving of drops, combining/merging drops, splitting drops, mixing drops, routing drops, and so on.

In some cases, the control system 140 considers the movement paths of multiple chemical inputs 115, or associated operations, to determine or select paths for the chemical inputs 115 with respect to one another. For example, the control system 140 may control the movement of drops to select shorter paths of movement when combining drops, to select paths of movement that do not cause possible collisions of drops as they move to a target electrode, and so on.

In some embodiments, the DMF device 120, via the control system 140, may perform operations to screen or otherwise characterize the chemical compounds 135. For example, the DMF device 120 may control the temperature (e.g., via a Peltier device) applied at a specific electrode upon which a chemical compound sits, to screen the compound using a polymerase chain reaction (PCR) operation.

As another example, the MF device 120 may apply a magnetic field at a specific electrode upon which an input or compounds sits, which may be attached to a nano-magnetic bead, to perform operations that purify and/or concentrate the chemical via the applied magnetic field. Further details regarding components of the DMF device 120 are described herein.

In addition to controlling the orchestration of drops across or over the electrode array 125, the control system 140 may also provide digital control to other components, such as the microchannels of the input system 110 and/or the chemical library 130.

In some embodiments, the DMF device 120 facilitates implementation of a chemical protocol for building the chemical library 130 as a DEL. The chemical protocol enables the synthesis of DNA-barcoded drug compounds by employing the DMF device 120 to join different and many combinations of small molecules. For example, the protocol can implement, via the DMF device 120, a “split” and “pool” approach, which includes attaching unique DNA barcode segments to different molecules in different droplets; pooling and mixing the molecules, splitting and joining different molecules into different droplets, and attaching unique DNA barcodes identifying the different molecules, and so on.

When pooling, each new molecule is joined to all previous combinations, in parallel. Accordingly, the number of distinct combinations that are assembled may scale exponentially with the number of joining steps: with n+n+ . . . +n steps, the DMF device 120 can assemble n×n . . . ×n combinations, leading to a large number (e.g., millions to billions) of distinct combinations. What follows is a large library of combinations, where each combination, or compound, has a unique strand of DNA—called a DNA “barcode”—attached. The DMF device 120 may then facilitate mixing the bar-coded molecules together and screening the molecules to see if any molecule or molecules bind to a target protein (e.g., by reading the barcode).

Further examples of operations performed by the DMF device 120 include DNA barcode synthesis on a chip (e.g., a parallel synthesis of DNA barcodes from a small set of “symbols”—small segments of DNA—via a sequence of hybridization and release of DNA strands), and/or DNA immobilization and release on a chip, with magnetic nanobeads.

Thus, in some embodiments, the technology described herein facilitates the synthesis of the chemical library 130 and/or the screening of the chemical compounds 135 within the chemical library 130. Such operations of synthesis and screening are used in drug discovery, where the chemical compounds 135 are screened for their ability to bind to a target protein, as well as other applications that seek to determine whether a compound can perform a certain function.

The DMF device 120, therefore, performs combinatorial chemistry to systematically combine smaller molecules, in many or all possible combinations, to create libraries of compounds. The DMF device 120, in some cases, can synthesize DNA-encoded libraries (DELs), where short DNA sequences are attached to all entries. These DNA sequences serve as unique barcodes, allowing for the efficient identification and tracking of these compounds in a mixture. DELs enable the simultaneous synthesis and screening of vast libraries of compounds.

In some cases, the DMF device 120 can synthesize peptide libraries, which include different sequences of amino acids, systematically joined in many or all possible combinations. Such peptides are the building-blocks of proteins, and a peptide library may be employed for drug discovery applications (e.g., identifying peptides that bind to therapeutic targets), epitope mapping (e.g., determining specific amino acid sequences recognized by antibodies), protein-protein interactions (e.g., learning how proteins interact with each other within cells), biomarker discovery (e.g., identifying peptides that are indicators of disease), and so on.

In some cases, the DMF device 120 may facilitate the screening of compounds, such as by performing high-throughput screening (HTS). HTS, used in drug discovery, tests and identifies active compounds, antibodies, or genes from chemical libraries. The DMF device 120 may perform automated testing of most or all entries in the library to see which bind to a target, to identify promising candidates for further research.

In some cases, the DMF device 120 may perform both synthesis and screening of combinatorial libraries, such as DELs and/or peptide libraries.

Thus, in various embodiments, the DMF device 120 may act to build chemical libraries for new applications and/or for existing operations, such as those that employ liquid-handling robotics. The DMF device 120 may receive the chemical inputs 115 via the mechanical, pressure-based, or acoustic-based handlers (e.g., in microliter volumes) and perform the synthesis in nanoliter volumes, replacing existing techniques that utilize robots for synthesis. The DMF device 120 may then provide the synthesized combinations back to mechanical handlers, which move the combinations to a drug library or other application.

FIG. 1 and the components, systems, servers, and devices depicted herein (e.g., the control system 140) provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

In some cases, the control system 140 may communicate with the DMF device 120 or other components via a network or cloud. The network or cloud can be any network, such as a wired or wireless local area network (LAN), a wired or wireless wide area network (WAN), the Internet or some other public or private network, a cellular (e.g., 4G, LTE, or 5G network), and so on. While the connections between the various devices and the network are shown as separate connections, these connections can be any kind of local, wide area, wired, or wireless network, public or private.

Further, any or all components depicted in the Figures described herein can be supported and/or implemented via one or more computing systems or servers. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, XR/AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of the DMF Device

As described herein, the technology utilizes a digital microfluidic (DMF) device to perform various operations, such as the synthesizing and/or screening of chemical libraries. The DMF device can be configured to operate on small droplets (e.g., ˜nanoliter or ˜picoliter sizes), to operate on many droplets at once (e.g., using a large grid of electrodes), and/or to operate quickly when moving, merging, splitting, mixing, or otherwise manipulating droplets during the synthesis/screening.

For example, a DMF device, such as the DMF device 120, may be configured to perform split and pool synthesis with a high throughput. Such a configuration may include certain electrode shapes or geometries, precise control of individual electrodes, thermal cycling at individual electrodes, magnetic field application at individual electrodes (or groups of electrodes), and so on.

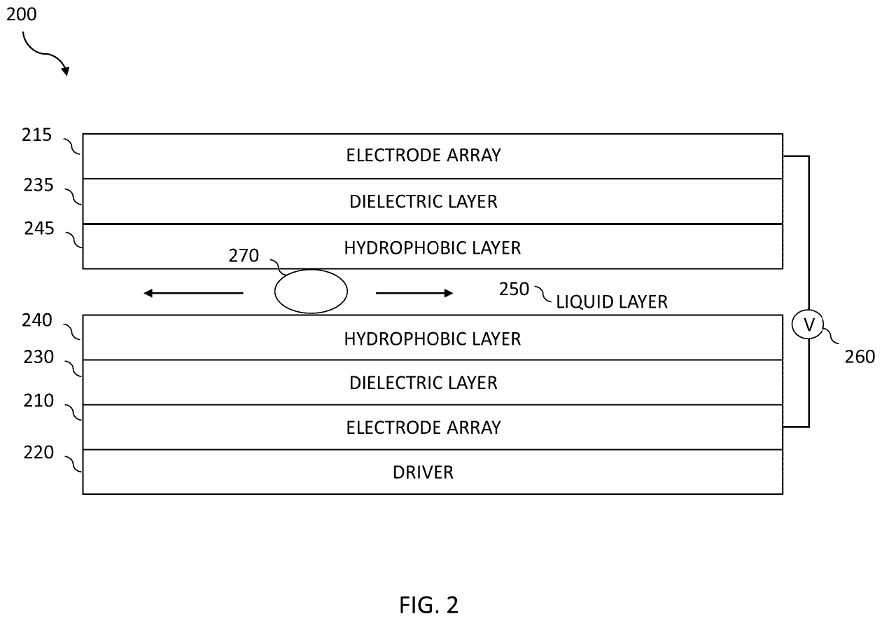

FIG. 2 is a block diagram illustrating an example digital microfluidic (DMF) device 200 for synthesizing chemical compounds from chemical inputs. The architecture of the DMF device 200 includes an electrode array layer 210, which contains an array of electrodes, as described herein.

The electrode array may be formed on a micro-scale or nano-scale and can include up to 1 million (1,000 by 1,000) electrodes. The electrode array can be formed of a conductive material, such as metal patterned on a substrate. In some cases, the electrode array layer 210 includes a substrate. In some cases, the substrate is a printed circuit board (PCB).

In some cases, the substrate is a silicon wafer or is formed of silicon. The silicon substrate can include a silicon interposer, which is a thin slice of silicon used to interface between a printed circuit board (PCB) below the silicon layer. The interposer can include a network of electrical pathways and micro-bumps or through-silicon vias (TSVs) for connections. The interposer may be precisely placed onto a driver layer 220 (e.g., a driver PCB) and electrically connected through solder balls or micro-bumps that align with the PCB's contact pads.

In some cases, the substrate is glass, and electrodes disposed on the glass substrate may be connected to the driver layer 220 beneath the glass with through-glass vias (TGVs).

The electrode array layer 210 is deposited, placed, or otherwise disposed onto the driver layer 220. The driver layer 220 may be a PCB, as described herein, and include advanced PCB technology with high layer counts (e.g., up to 40), aligned with precision. The driver layer 220 may include materials that limit certain effects (e.g., out-gassing), such as high-Tg epoxy resins, polyimide, PTFE (Teflon), and ceramics. The driver layer 220 may be formed of various fabrication techniques, such as fine-line technology laser drilling, microvias, and/or sequential lamination.

In some cases, the driver layer 220 includes a transistor, such as a thin-film transistor (TFT) backplane. The TFT backplane may serve as a base for the array of individually controllable electrodes that manipulate droplets during various operations. The TFTs are electrically connected to their corresponding electrodes, grouping electrodes into rows and columns. The voltage at any individual electrode can be turned on or off by activating a corresponding row and column. Thus, the DMF device can employ an addressing scheme that uses an M+N connection for M columns and N row DMF grids.

A dielectric layer 230 is deposited on the electrode array layer 210. The dielectric layer 230, which acts as an isolation or insulating layer, may be formed of aluminum oxide, hafnium oxide, zirconium oxide, hafnium zirconium oxide, titanium oxide, scandium oxide, yttrium oxide, lanthanum oxide, lutetium oxide, niobium(v) oxide, tantalum(v) oxide, a fluoropolymer, or various combinations.

A hydrophobic layer 240 is deposited on the dielectric layer 230. The hydrophobic layer 240 provides a hydrophobic surface, or liquid layer 250, upon which a drop 270 (or drops) sits or moves. The hydrophobic layer 240 may be formed of polytetrafluoroethylene (PTFE) or Teflor, Cytop, silicones, perfluorinated compounds, parylene, hydrophobic silane coatings, diamond-like carbon (DLC), and so on.

In some cases, the hydrophobic layer 240 is formed of a hydrophobic microstructure and deposited (e.g., micro-machined or deposited via CVD) via photo-patterning and wet etching, photo-patterning and dry etching, by a photoresist lift off after deposition, by laser micro-machining, by laser induced wet etching, by laser induced dry etching, and so on. In some cases, the hydrophobic layer 240 and/or the dielectric layer 230 may be replaced by a hybrid or single dielectric/hydrophobic layer that includes some or all the materials described herein.

As described herein, the drop 270 may move within or through the liquid layer 250, which is an area above the hydrophobic layer 240. In some cases, a voltage 260 is applied directly to the drop 270 to facilitate movement via electrowetting. However, in some embodiments, the DMF device 200 includes a top plate, which acts as a ceiling or upper boundary of the liquid layer 250 and applies electrowetting forces to a top of the drop 270 (in addition to forces applied below via the electrode array).

The top plate, in some cases, can include a transparent material or plate, and may have a hydrophobic layer 245, dielectric layer 235, and/or electrode array layer 215. The hydrophobic layer 245 may be like the hydrophobic layer 240, the dielectric layer 235 may be like the dielectric layer 230, and the electrode array layer 215 may be like the electrode array layer 210.

The top plate may enclose the liquid layer 250, and the voltage may drive the electrode array (or another conductive layer that acts as a grounding plane). Thus, the top plate may include the hydrophobic layer 245, dielectric layer 235, and/or electrode array layer 215, and a bottom plate may include the driver layer 220, the electrode array layer 210, the dielectric layer 230, and the hydrophobic layer 240. The liquid layer 250, therefore, may be enclosed by the bottom plate and the top plate.

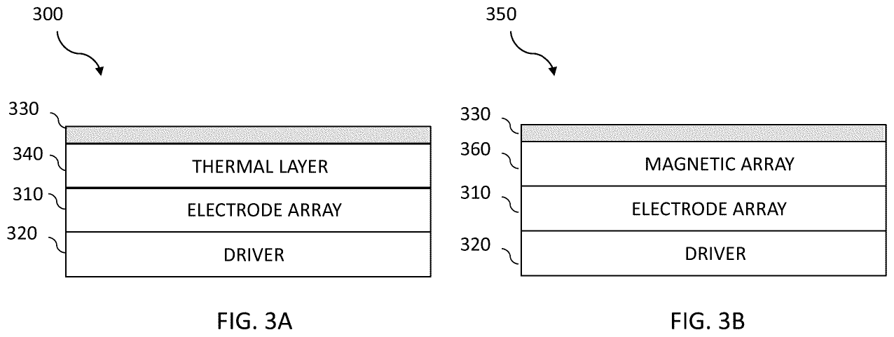

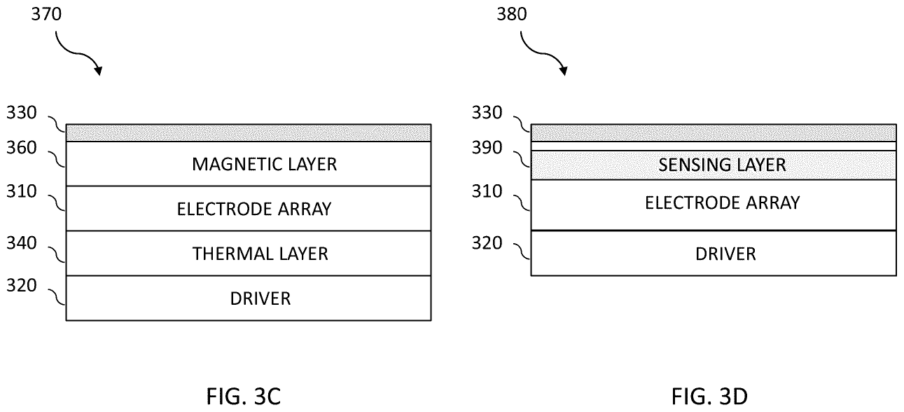

Of course, the DMF device 200 can include other layers or components. FIGS. 3A-3D are block diagrams that illustrate different layers of an example DMF device.

FIG. 3A depicts a DMF device 300 that includes an electrode array layer 310, a driver layer 320, a hydrophobic and/or dielectric layer 330, and a thermal layer 340. The thermal layer 340 may include components, such as heating and/or cooling elements, that maintain, heat, and/or cool an electrode (or group of electrodes) to a certain temperature. In some cases, the thermal layer is provided above or proximate the electrode array layer 310 (as shown). In some cases, the thermal layer 340 may be placed, formed, or disposed at other locations, such as part of the hydrophobic and/or dielectric layer 330.

For example, the thermal layer 340 may provide and/or include a Peltier device and temperature sensors. The Peltier device, or a similar thermoelectric module, provides thermoelectric cooling or heating at an electrode or electrodes (e.g., an array of electrodes contained in the electrode array layer 310). The sensors can measure a temperature at the electrode or electrodes and provide information to the control system 240 or another monitoring system. The control system 240, therefore, can include a dynamic feedback system that adjusts or modifies an applied temperature at an electrode or electrodes.

FIG. 3B depicts a DMF device 350 that includes a magnetic layer 360. The magnetic layer 360 can include magnetic elements, which can control, magnetically, nanoparticles (e.g., magnetic nanobeads) at or associated with electrodes, such as during the screening of chemical compounds at the electrodes or other solid-phase chemistry applications. In some cases, the magnetic layer 340 may be placed, formed, or disposed at other locations, such as part of the hydrophobic and/or dielectric layer 330.

The magnetic elements may include electro-magnets, permanent magnets, micro-coils, planar coils, ferrofluids, Microelectromechanical systems (MEMS), Halbach arrays, and so on. Further, the magnetic elements may include magnetic bead mixtures that are dried and disposed on or within the array of electrodes (and released into a solution by introducing a buffer solution and turning off an applied magnetic field), and/or may be dispensed via the input channels or tubes described herein.

In some cases, the magnetic layer 360 enables precise control of a magnetic field at one or more designated electrodes. Such control can enable the DMF device 350 to immobilize DNA attached to magnetic nanobeads (e.g., during purifying and/or concentrating the contents of droplets), among other actions.

FIG. 3C depicts a DMF device 370 that includes a magnetic layer 360 and a thermal layer. The DMF device 370 may employ both layers during certain operations, such as operations that screen chemical compounds during reactions or other chemical operations. In some cases, the layers may be placed, formed, or disposed at other locations, such as part of the hydrophobic and/or dielectric layer 330.

FIG. 3D depicts a DMF device 380 that includes a sensing layer 390. The sensing layer 390 can include chemical sensing elements that sense or capture information associated with drops/droplets. In some cases, the sensing elements can be:

Capacitive sensors that provide an electronic readout that indicates whether there is a droplet on an electrode and/or an electrical readout of the chemical contents of the droplet;

Biosensor field-effect transistors for detecting the chemical contents of droplets and quantifying binding events. These BIOFETs can be disposed as a separate layer and/or integrated throughout the DMF device 390 or 200 (e.g., into a substrate or a top plate cover);

Elements for enzyme-linked immunosorbent assay (ELISA) detecting chemical reaction and binding events. These elements can be disposed as a separate layer and/or integrated throughout the DMF device 390 or 200 (e.g., into the dielectric layer 330); and so on.

As shown, the sensing layer 390 can be part of the electrode array 310. In some cases, sensing elements may be placed, formed, or disposed at other locations, such as part of the hydrophobic and/or dielectric layer 330 and/or within a liquid layer.

Further, a DMF device, such as the DMF devices 200, 300, 350, 370, 380 may include optical or photonic sensing elements. These elements may capture images (e.g., for fluorescent or optical readouts of the chemical contents of droplets). In some cases, a DMF device may include a camera or optical sensor above or within the liquid layer 250, which captures images/video of drop/droplet movement across an array of electrodes.

As described herein, an electrode array layer (e.g., the electrode array layer 210) provides an array of electrodes (or electrode array) that includes multiple electrodes disposed in a grid or other pattern. The electrodes may be addressed separately, with each electrode having an applied voltage (on or off) during electrowetting orchestration of drops or other operations.

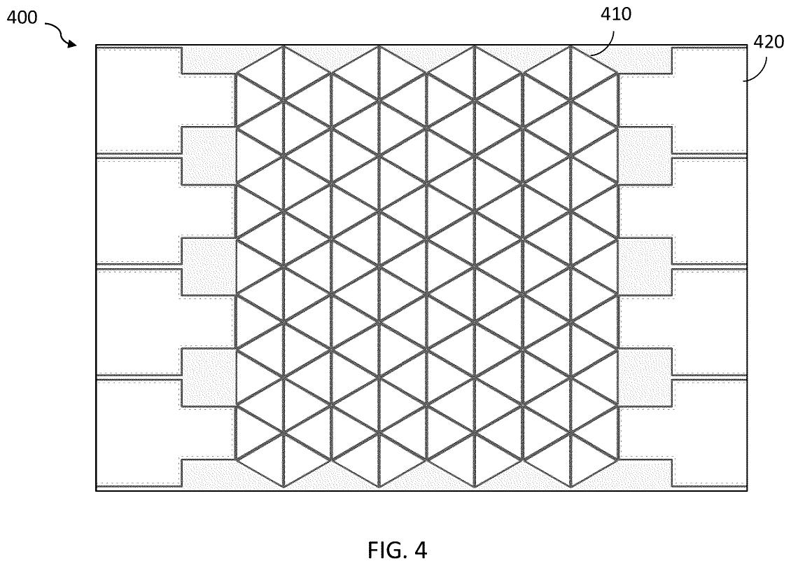

FIG. 4 is a diagram illustrating an array of electrodes 400 for use with a DMF device. The array of electrodes 400 includes multiple different electrodes 410 and edge electrodes 420, which may be larger electrodes configured to receive droplets/drops dispensed to the array of electrodes (e.g., to an area, such as a liquid layer, that is above the array of electrodes and separated by one or more layers, such as a hydrophobic layer). For example, in some cases, the edge electrodes 420 are physically coupled and/or proximate to microchannels that supply liquids to the array of electrodes 400.

As shown, each electrode 410 has a triangular shape. Such a geometrical shape can provide certain benefits at small feature sizes. For example, triangles have the longest adjacent-side length with respect to each other (for a given shape area of any shape that can tesselate). Further, given that the electrowetting force pulling a droplet from one electrode to another is proportional to the adjacent side length, the use of triangular electrodes can increase the reliability of movement, splitting, and/or merging operations, and decrease the applied voltage effecting these operations. Thus, the use of a triangle for the shape of the electrode 410 can provide effective and/or suitable droplet movement and/or droplet splitting during operations, among other benefits.

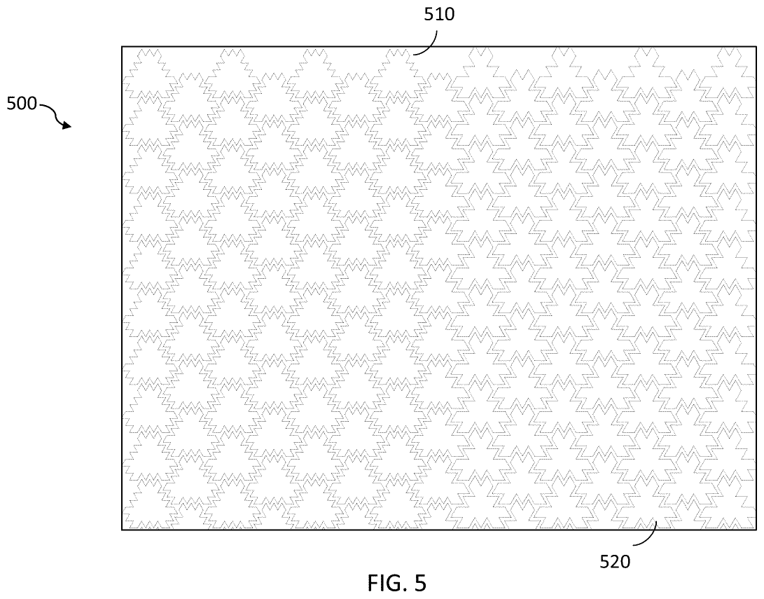

FIG. 5 is a diagram illustrating another array of electrodes 500 for use with a DMF device. The array of electrodes 500 includes a first set of electrodes 510 and a second set of electrodes 520, each having different shapes or geometries (e.g., hexagonal or snowflake shapes having large boundary spans). Thus, an array of electrodes may include or be tessellated by electrodes of different shapes, sizes, or configurations, which can provide for specific movement or splitting characteristics of drops or other liquids. Further, the size or shape of the electrodes can be tailored to the size of the drops being manipulated and/or the number of drops being manipulated at any given time or operation.

In addition, the use of certain shapes may lower the operating voltage for applying voltages to the electrodes, and facilitates independent, precise control of voltage at each electrode (e.g., using AC, DC, and/or sawtooth waveforms).

### Examples of Performing Operations Using a DMF Device

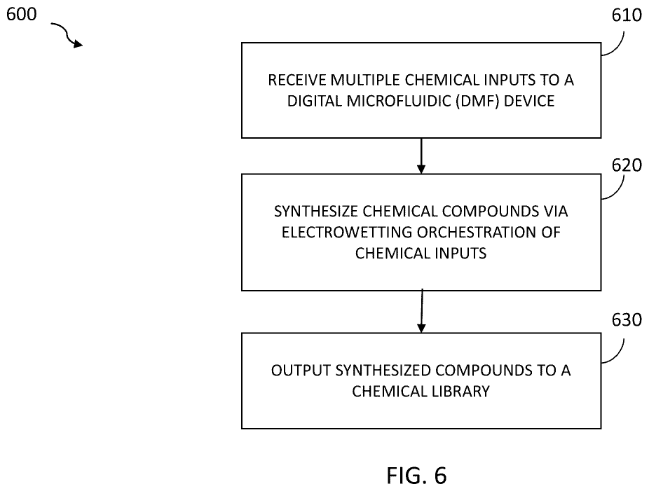

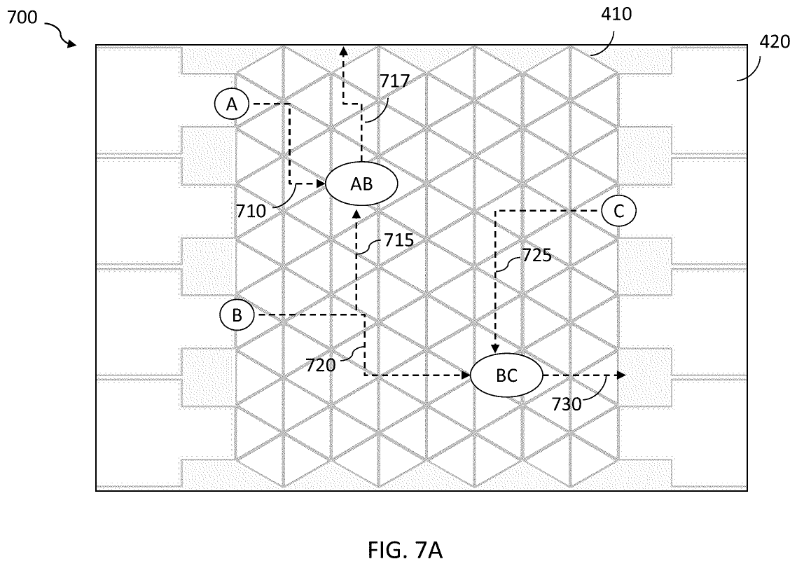

In some embodiments, as described herein, the technology facilitates the creation of chemical libraries using a DMF device. FIG. 6 is a flow diagram illustrating a method 600 for creating a chemical library using a DMF device. The method 600 may be performed by the DMF device 120 and, accordingly, is described herein merely by way of reference thereto. It will be appreciated that the method 600 may be performed on any suitable hardware.

In operation 610, the DMF device 120 receives multiple chemical inputs. For example, the DMF device 120 receives a set of chemical inputs 115 via the input system 110, which dispenses the chemical inputs via microchannels to the DMF device 120.

In operation 620, the DMF device 120 synthesizes multiple chemical compounds via electrowetting orchestration of the multiple chemical inputs. For example, the DMF device 200, via the control system 140, may cause the chemical inputs to move to certain electrodes, where they are combined into chemical compounds. As another example, the DMF device 200 may cause the splitting and later combining of the chemical inputs at certain electrodes and/or along certain paths of electrodes.

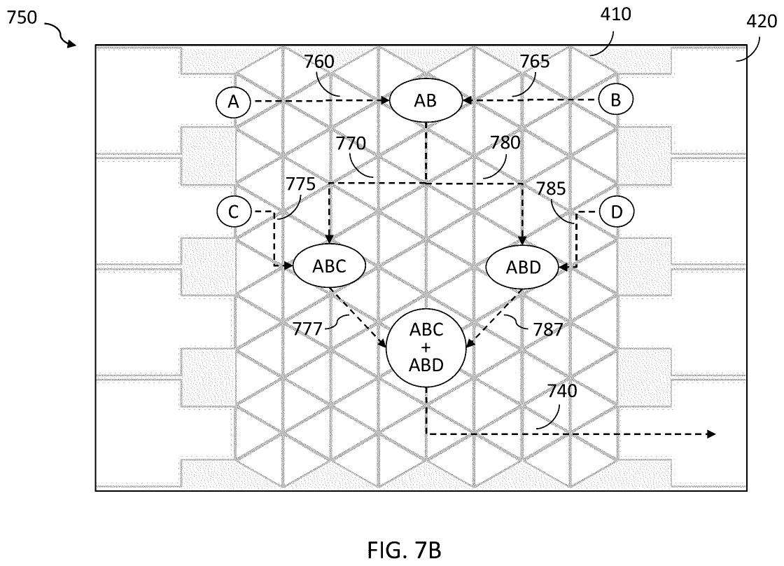

FIG. 7A is a diagram illustrating an electrowetting orchestration 700 of chemical inputs using a DMF device. As shown, an input A follows a path 710 to a determined electrode. An input B follows a path 715 to the same electrode. At the electrode, the input A and input B combine to form a chemical compound AB, which travels along a path 717 out of the DMF device and to a chemical library. On the same DMF device, the input B is split and travels a path 720 to another electrode. An input C travels a path 725 to that electrode, and a compound BC is synthesized. The compound BC follows path 730 out of the DMF device.

FIG. 7B is a diagram illustrating an electrowetting orchestration 750 of chemical inputs using a DMF device. As shown, an input A follows a path 760 to a determined electrode. An input B follows a path 765 to the same electrode. At the electrode, the input A and input B combine to form a chemical compound AB. Compound AB is split and travels along a path 770 to a new electrode. An input C travels a path 775 to that electrode, and a compound ABC is synthesized. On the same DMF device, the split compound AB travels a path 780 to another electrode. An input D travels a path 785 to that electrode, and a compound ABD is synthesized. Compound ABD then follows a path 787 to a new electrode. Simultaneously, compound ABC follows a path 777 to the same electrode to combine with ABD to form a library of compounds ABC+ABD. The library of compounds ABC+ABD follows a path 790 out of the DMF device.

Back to FIG. 6, in operation 630, the DMF device 120 outputs the synthesized multiple chemical compounds to a chemical library that is coupled to the DMF device 120. For example, the DMF device, as shown in FIG. 7, causes a newly synthesized compound to exit to a chemical library, such as a DEL.

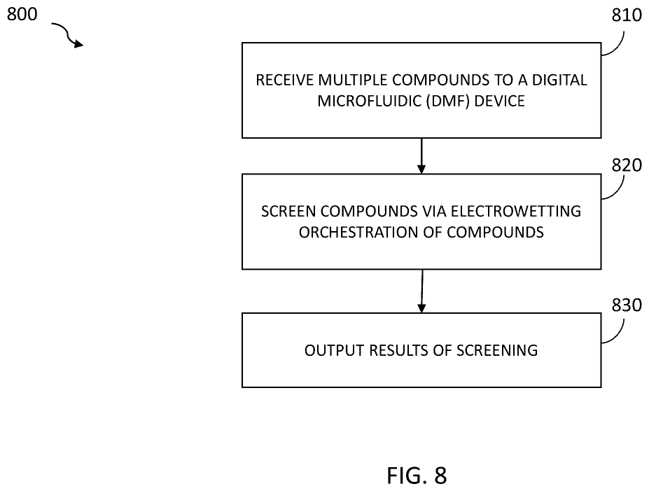

In some embodiments, as described herein, the technology facilitates the screening of chemical compounds. FIG. 8 is a flow diagram illustrating a method 800 for screening chemical compounds using a DMF device. The method 800 may be performed by the DMF device 120 and, accordingly, is described herein merely by way of reference thereto. It will be appreciated that the method 800 may be performed on any suitable hardware.

In operation 810, the DMF device 120 receives multiple chemical compounds, such as those created by the DMF device 120. For example, the DMF device 120 receives a set of chemical compounds 135 via the chemical library 130, which dispenses the chemical compounds 130 via microchannels to the DMF device 120.

In operation 820, the DMF device 120 screens the compounds using electrowetting orchestration. For example, the DMF device 120, via the control system 140, may cause the chemical compounds to move to certain electrodes, where certain reactions or screening operations occur or commence.

As described herein, a screening operation can include various processes or reactions. Example operations can include operations directed to the quality control of synthesized compounds (e.g., liquid chromatography mass spectrometry, or LCMS), hit detection (e.g., PCR of DNA tags), and so on.

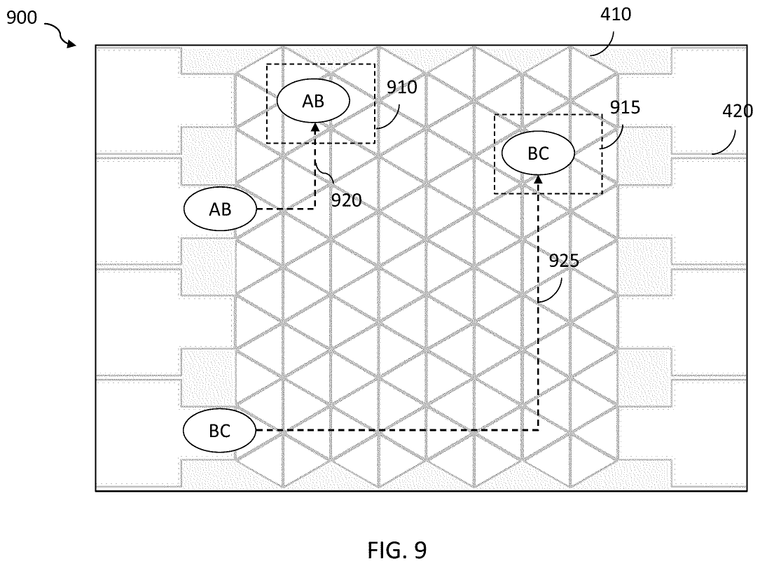

FIG. 9 is a diagram illustrating an electrowetting orchestration 900 of chemical compounds using a DMF device. As shown, a first compound AB follows a path 920 to a target zone 910 of electrodes, such as a set of electrodes configured to perform a PCR operation. Similarly, a second compound BC travels via a path 925 to a target zone 915 of electrodes, such as a set of electrodes configured to screen the compound BC to determine whether it binds to a target protein (e.g., by reading a DNA barcode attached to the compound BC).

Back to FIG. 8, in operation 830, the DMF device 120 outputs the results of the screening. For example, the DMF device 120, via the control system 140 or an associated computing system, may transmit a report or other data that provides information regarding the screening.

As described herein, the DMF device 120 can screen compounds before they are offloaded to a library and/or compounds loaded back onto the DMF device 120. Thus, in some cases, the DMF device 120 can perform screening operations for compounds synthesized by the DMF device 120 and/or compounds newly provided to the DMF device 120.

Thus, in various embodiments, a DMF device is configured and/or adapted to synthesize chemical inputs and/or screen chemical compounds using electrowetting propulsion over an array of electrodes. The DMF device may be configured to have a specific layout of electrodes to perform certain operations (e.g., split and pool synthesis) and/or to orchestrate large numbers of combinations or operations, among other benefits.

### Examples of the Disclosed Technology

The technology described herein may be provided and/or implemented as one or more embodiments, including the following.

In some embodiments, an apparatus for synthesizing a chemical library includes an array of electrodes configured to move drops containing chemical inputs using electrowetting propulsion, multiple microchannels positioned to dispense the drops containing the chemical inputs to an area above the array of electrodes, and multiple microchannels positioned to receive chemical compounds synthesized using within the area above the array of electrodes.

In some cases, the array of electrodes is part of a digital microfluidic (DMF) device that includes a bottom plate, having a driver layer, an electrode array layer, which includes the array of electrodes, disposed on the driver layer, a dielectric layer disposed on the electrode array layer, and a hydrophobic layer disposed on the dielectric layer.

In some cases, the apparatus includes a top plate disposed above the bottom plate, wherein the top plate includes a top electrode array layer that includes a top array of electrodes, a top dielectric layer disposed below the top electrode array layer, and a top hydrophobic layer disposed below the top dielectric layer, wherein a top surface of the hydrophobic layer and a bottom surface of the top hydrophobic layer form a channel that surrounds the area above the array of electrodes of the bottom plate.

In some cases, the apparatus includes a thermal layer configured to control a temperature at the area above the array of electrodes.

In some cases, the apparatus includes a magnetic layer configured to apply a magnetic field to the area above the array of electrodes.

In some cases, the apparatus includes a sensing layer configured to sense information associated with the drops containing the chemical inputs.

In some cases, the driver layer includes a printed circuit board (PCB) layer or a transistor layer.

In some cases, the dielectric layer is formed of aluminum oxide, hafnium oxide, zirconium oxide, hafnium zirconium oxide, titanium oxide, scandium oxide, yttrium oxide, lanthanum oxide, lutetium oxide, niobium(v) oxide, tantalum(v) oxide, a fluoropolymer, or combinations thereof.

In some cases, the hydrophobic layer is formed of polytetrafluoroethylene (PTFE), Cytop, silicone, a perfluorinated compound, parylene, a hydrophobic silane coating, or diamond-like carbon (DLC).

In some cases, the the hydrophobic layer is a combined hydrophobic and dielectric layer.

In some cases, the apparatus includes a top plate is placed above the hydrophobic layer to form a liquid layer that includes the area above the array of electrodes.

In some cases, the array of electrodes includes multiple electrodes having a triangular shape.

In some cases, the array of electrodes includes multiple electrodes having a hexagonal shape.

In some cases, the apparatus includes a microplate that contains the chemical inputs, wherein the microchannels positioned to dispense the drops containing the chemical inputs to an area above the array of electrodes are physically coupled to the microplate.

In some cases, the apparatus includes an optical sensor disposed above or within the area above the array of electrodes.

In some embodiments, a method includes receiving multiple chemical inputs to a digital microfluidics (DMF) device that includes an array of electrodes, synthesizing multiple chemical compounds via electrowetting orchestration of the multiple chemical inputs within the DMF device, and outputting the synthesized multiple chemical compounds to a chemical library that is physically coupled to the DMF device.

In some cases, the electrowetting orchestration of the multiple chemical inputs includes combining a first set of chemical inputs into a first chemical compound at a first electrode of the array of electrodes and combining a second set of chemical inputs into a second chemical compound at a second electrode of the array of electrodes.

In some cases, the electrowetting orchestration of the multiple chemical inputs includes combining a first set of chemical inputs into a first chemical compound at a first electrode of the array of electrodes, combining a second set of chemical inputs into a second chemical compound at a second electrode of the array of electrodes, and combining the first chemical compound and the second chemical compound into a third chemical compound at a third electrode of the array of electrodes.

In some cases, the chemical library is a DNA-encoded library (DEL), and wherein synthesizing the multiple chemical compounds includes attaching a short DNA sequence to each of the chemical compounds.

In some embodiments, a system for synthesizing a DNA-encoded library (DEL) includes a loading module containing microfluidic channels that dispense chemical inputs contained in a microplate, a digital microfluidic (DMF) device that is physically coupled to the loading module and configured to receive the dispensed chemical inputs, combine the chemical inputs via electrowetting propulsion to form chemical compounds that are tagged with a short DNA sequence, and screen the chemical compounds, and a library module coupled to the DMF device that receives the chemical compounds from the DMF device.

In some embodiments, a system for synthesizing a peptide library includes a loading module containing microfluidic channels that dispense chemical inputs contained in a microplate, a digital microfluidic (DMF) device that is physically coupled to the loading module and configured to receive the dispensed chemical inputs, combine the chemical inputs via electrowetting propulsion to form chemical compounds, and screen the chemical compounds, and a library module coupled to the DMF device that receives the chemical compounds from the DMF device.

### Conclusion

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. An apparatus for synthesizing a chemical library, the apparatus comprising:
an array of electrodes configured to move drops containing chemical inputs using electrowetting propulsion;
multiple microchannels positioned to dispense the drops containing the chemical inputs to an area above the array of electrodes; and
multiple microchannels positioned to receive chemical compounds synthesized using within the area above the array of electrodes.

2. The apparatus of claim 1, wherein the array of electrodes is part of a digital microfluidic (DMF) device that includes a bottom plate, having:
a driver layer;
an electrode array layer, which includes the array of electrodes, disposed on the driver layer;
a dielectric layer disposed on the electrode array layer; and
a hydrophobic layer disposed on the dielectric layer.

3. The apparatus of claim 2, further comprising:
a top plate disposed above the bottom plate, wherein the top plate includes:
a top electrode array layer that includes a top array of electrodes;
a top dielectric layer disposed below the top electrode array layer; and
a top hydrophobic layer disposed below the top dielectric layer,
wherein a top surface of the hydrophobic layer and a bottom surface of the top hydrophobic layer form a channel that surrounds the area above the array of electrodes of the bottom plate.

4. The apparatus of claim 2, further comprising:
a thermal layer configured to control a temperature at the area above the array of electrodes.

5. The apparatus of claim 2, further comprising:
a magnetic layer configured to apply a magnetic field to the area above the array of electrodes.

6. The apparatus of claim 2, further comprising:
a sensing layer configured to sense information associated with the drops containing the chemical inputs.

7. The apparatus of claim 2, wherein the driver layer includes a printed circuit board (PCB) layer or a transistor layer.

8. The apparatus of claim 2, wherein the dielectric layer is formed of aluminum oxide, hafnium oxide, zirconium oxide, hafnium zirconium oxide, titanium oxide, scandium oxide, yttrium oxide, lanthanum oxide, lutetium oxide, niobium(v) oxide, tantalum(v) oxide, a fluoropolymer, or combinations thereof.

9. The apparatus of claim 2, wherein the hydrophobic layer is formed of polytetrafluoroethylene (PTFE), Cytop, silicone, a perfluorinated compound, parylene, a hydrophobic silane coating, or diamond-like carbon (DLC).

10. The apparatus of claim 2, wherein the hydrophobic layer is a combined hydrophobic and dielectric layer.

11. The apparatus of claim 2, further comprising:
a top plate is placed above the hydrophobic layer to form a liquid layer that includes the area above the array of electrodes.

12. The apparatus of claim 1, wherein the array of electrodes includes multiple electrodes having a triangular shape.

13. The apparatus of claim 1, wherein the array of electrodes includes multiple electrodes having a hexagonal shape.

14. The apparatus of claim 1, further comprising:
a microplate that contains the chemical inputs, wherein the microchannels positioned to dispense the drops containing the chemical inputs to an area above the array of electrodes are physically coupled to the microplate.

15. The apparatus of claim 1, further comprising:
an optical sensor disposed above or within the area above the array of electrodes.

16. A method, comprising:
receiving multiple chemical inputs to a digital microfluidics (DMF) device that includes an array of electrodes;
synthesizing multiple chemical compounds via electrowetting orchestration of the multiple chemical inputs within the DMF device; and
outputting the synthesized multiple chemical compounds to a chemical library that is physically coupled to the DMF device.

17. The method of claim 16, wherein the electrowetting orchestration of the multiple chemical inputs includes:
combining a first set of chemical inputs into a first chemical compound at a first electrode of the array of electrodes; and
combining a second set of chemical inputs into a second chemical compound at a second electrode of the array of electrodes.

18. The method of claim 17, wherein the electrowetting orchestration of the multiple chemical inputs includes:
combining a first set of chemical inputs into a first chemical compound at a first electrode of the array of electrodes;
combining a second set of chemical inputs into a second chemical compound at a second electrode of the array of electrodes; and
combining the first chemical compound and the second chemical compound into a third chemical compound at a third electrode of the array of electrodes.

19. The method of claim 17, wherein the chemical library is a DNA-encoded library (DEL), and wherein synthesizing the multiple chemical compounds includes attaching a short DNA sequence to each of the chemical compounds.

20. A system for synthesizing a DNA-encoded library (DEL), the system comprising:
a loading module containing microfluidic channels that dispense chemical inputs contained in a microplate;
a digital microfluidic (DMF) device that is physically coupled to the loading module and configured to:
receive the dispensed chemical inputs;
combine the chemical inputs via electrowetting propulsion to form chemical compounds that are tagged with a short DNA sequence; and
screen the chemical compounds; and
a library module coupled to the DMF device that receives the chemical compounds from the DMF device.
